(19) United States
(12) Patent Application Publication
(10) Pub. No.: US 2025/0262079 A1
(43) Pub. Date: Aug. 21, 2025

(54) BLACK BOX JOINT BRACE

(71) Applicant: Steven M. Greenberg, BOYNTON BEACH, FL (US)

(72) Inventor: Steven M. Greenberg, BOYNTON BEACH, FL (US)

(21) Appl. No.: 18/583,840

(22) Filed: Feb. 21, 2024

(51) Int. Cl. A61F 5/01; A61B 5/00; A61B 5/11; G16H 40/67

(52) U.S. Cl. CPC A61F 5/0109; A61B 5/1114; A61B 5/6812; A61F 5/0118; G16H 40/67; A61B 2560/0214

(57) ABSTRACT

A black box joint brace incudes a flexible sleeve such as a neoprene sleeve defining a hollow channel with a circumference that is less than a circumference of a target limb encapsulating a mammalian joint such as a knee joint, elbow joint, wrist joint or ankle joint. The brace additionally includes a power source fixed to the sleeve and a set of positional sensors disposed opposite to one another on the sleeve and sensing a spatial position of each of different limbs positioned oppositely across the mammalian joint. Finally, the joint brace includes memory powered by the power source and storing the spatial position sensed by the set of positional sensors.

### BACKGROUND OF THE INVENTION

### Field of the Invention

The present invention relates to the field of joint braces.

### Description of the Related Art

Joint bracing refers to support articles of clothing one wears for a joint perceived to be perceptible to injury or re-injury. Joint braces generally include knee braces, elbow braces, wrist braces and ankle braces enveloping the knee joint, elbow joint, wrist joint and ankle joint, respectively. The typical joint brace provides an elastic quality with a spring coefficient intended to bias the limbs about a joint to a straight position and to apply compression force to the joint. Joint braces are frequently formed from combinations of metal, foam, plastic, elastic material and straps and joint braces can be found in many sizes, colors and designs. While some wear joint braces for the purpose of protecting an injured joint, others wear joint braces in anticipation of a prospective injury with the aim of buttressing the joint to avoid injury.

Joint braces for the most part are passive devices reacting to the kinetics of the wearer. However, joint braces are unable to provide benefit beyond the immediate reflexive force applied to the joint and the connective ligaments. As such, despite the donning of a joint brace, the wearer is unable at a later time to recognize the consequence of the kinetic movements while wearing the joint brace. The consequence of such kinetic movements, however, can be essential in the diagnosis of an injury potentially incurred while wearing the joint brace. In this regard, presently, the diagnosis of an injury largely depends upon the physical inspection of the joint by a skilled health practitioner after the occurrence of an injury to the joint, or through imagery including MRI or X-ray imagery acquired long after the occurrence of the injury. In most instances, the health care practitioner receives only a subjective description from the patient as to the nature of the motion of the limbs relative to the joint at the time of injury.

### BRIEF SUMMARY OF THE INVENTION

Embodiments of the present invention address deficiencies of the art in respect to joint injury diagnosis and provide a novel and non-obvious black box joint brace. In an embodiment of the invention, a black box joint brace incudes a flexible sleeve such as a neoprene sleeve defining a hollow channel with a circumference that is less than a circumference of a target limb encapsulating a mammalian joint such as a knee joint, elbow joint, wrist joint or ankle joint. The brace additionally includes a power source fixed to the sleeve and a set of positional sensors disposed opposite to one another on the sleeve and sensing a spatial position of each of different limbs positioned oppositely across the mammalian joint. Finally, the joint brace includes memory powered by the power source and storing the spatial position sensed by the set of positional sensors.

In one aspect of the embodiment, the spatial position is a position of a lower one of the limbs relative to the mammalian joint. In another aspect of the embodiment, the spatial position is a position of an upper one of the limbs relative to the mammalian joint. In yet another aspect of the embodiment, the spatial position is a position of a lower one of the limbs relative to an upper one of the limbs.

Of note, the device additionally can include a rotary motion sensor powered by the power source and adapted to measure position, velocity and acceleration and to write the measured position, velocity and acceleration to the memory. Likewise, the device additionally can include a pulsometer fixed to an interior portion of the flexible sleeve and powered by the power source and adapted to measure a pulse through skin contact and to write the pulse to the memory. Even further, the device additionally can include a temperature sensor fixed to an interior portion of the flexible sleeve and powered by the power source and adapted to measure of a temperature of the mammalian joint through skin contact and to write the temperature to memory.

Optionally, the device includes an embedded computing system powered by the power source. Further, the device includes firmware executing in the embedded computing system and including program instructions to read the sensed spatial position from the set of positional sensors and to write the sensed spatial position to the memory. In this aspect, a shortwave radio frequency transmitter is provided, and controlled by the embedded computing system and configured to transmit the spatial position sensed by the set of positional sensors to a coupled computing device such as a smart watch or a mobile phone.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a black box joint brace adapted for generating video clip imagery of the relative movement of one or more limbs about a joint sensed by sensors on the joint brace;

FIG. 2 is a schematic illustration of a data processing system adapted to generate video clip imagery of the relative movement of one or more limbs about a joint sensed by sensors on a joint brace; and,

FIG. 3 is a flow chart illustrating a process for generating video clip imagery of the relative movement of one or more joint limbs about a joint sensed by sensors on a joint brace.

### DETAILED DESCRIPTION OF THE INVENTION

Embodiments of the invention provide for a black box joint brace. In accordance with an embodiment of the invention, a black box joint brace includes a joint brace such as knee brace, elbow brace, ankle brace or wrist brace that envelops two different portions of a limb of an end user opposite a joint juxtaposed therebetween. At least two sensors are embedded in the joint brace and each one of the sensors is adapted to sense a position of a corresponding limb to which the one of the sensors is proximate. The joint brace includes wireless communications circuitry powered by an onboard power source such as a batter and is adapted to transmit positional data sets from the sensors in association with different timestamps. Thereafter, the positional data sets for all recorded timestamps are processed to generate different visualizations of a relative position of the limbs relative to the joint at each timestamp and the visualizations are combined to form a video clip. The video clip is then transmitted to a recipient health care practitioner associated with the end user.

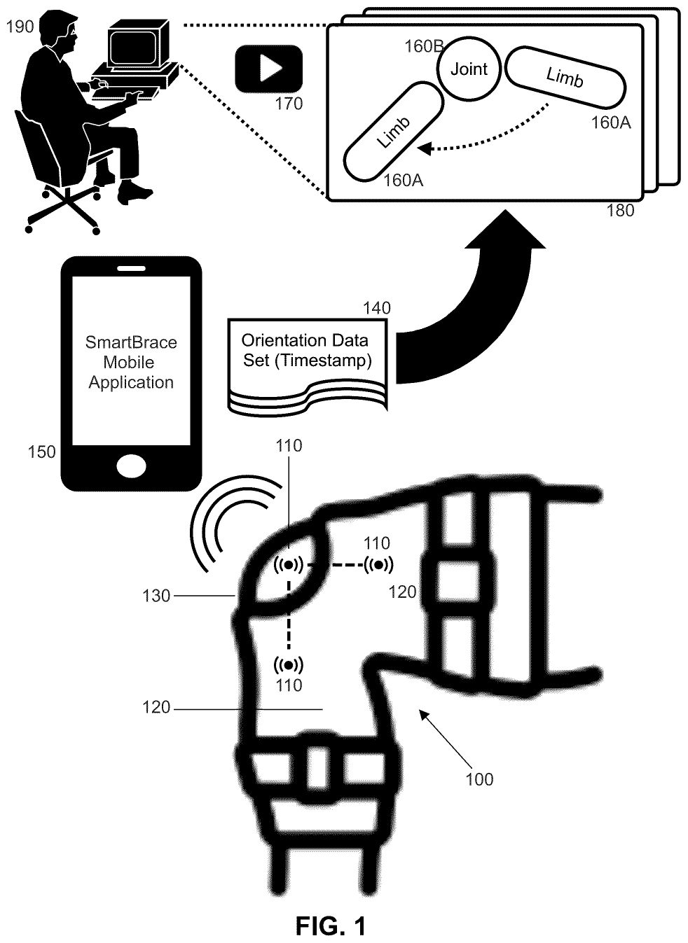

In further illustration, FIG. 1 pictorially shows a black box joint brace adapted for generating video clip imagery of the relative movement of one or more limbs about a joint sensed by sensors on the joint brace. As shown in FIG. 1, a smart brace 100 includes different positional sensors 110 oriented on the brace 100 at different known locations relative to one another. Each of the sensors 110 is spatially associated with a portion 120 of the limbs or a joint 130 disposed therebetween. A data set 140 of the position in space of each of the sensors 110 in association with a corresponding portion 120 or joint 130 of the limbs is then recorded at different timestamps and transmitted to a mobile application executing in smart phone 150. The mobile application 150 in turn uploads the data set 140 for each of the timestamps to a cloud service which generates a visualization 180 for each timestamp showing a relative position of each portion of the limbs 160A relative to the joint 160B. The cloud service then aggregates the visualizations 180 into a video clip 170 viewable by a health care practitioner and demonstrative of the movement of the limbs 120 relative to the joint 130 over a time span reflective of the timestamps of the visualizations 180.

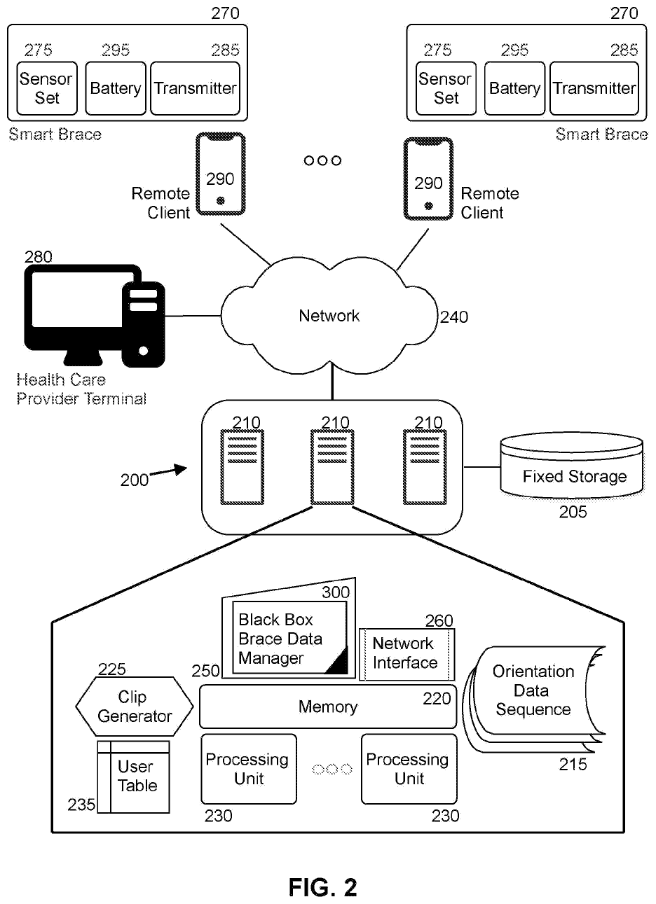

In further illustration, FIG. 2 schematically shows a data processing system adapted to generate video clip imagery of the relative movement of one or more limbs about a joint sensed by sensors on a joint brace. In the data processing system illustrated in FIG. 1, a host computing platform 200 is provided. The host computing platform 200 includes one or more computers 210, each with memory 220 and one or more processing units 230. The computers 210 of the host computing platform (only a single computer shown for the purpose of illustrative simplicity) can be co-located within one another and in communication with one another over a local area network, or over a data communications bus, or the computers can be remotely disposed from one another and in communication with one another through network interface 260 over a data communications network 240.

The host computing platform 200 is communicatively coupled to different remote computing clients 290, for instance different smart phones, smart watches or tablet computers, from over the data communications network 240. Each of the remote clients 290 enjoys a short range radio frequency communicative coupling to a corresponding smart brace 270. Each smart brace 270 includes at least a set 275 of different positional sensors which may include any combination of a rotary motion sensor, a temperature sensor and a pulsometer. As well, each smart brace 270 includes a power source powering the sensors in the sensor set 275. Finally, each smart brace includes a transmitter adapted to transmit sensed data from the sensors of the sensor set 275 over the short range radio frequency communicative coupling to an associated one of the remote clients 290.

Notably, a computing device 250 including a non-transitory computer readable storage medium can be included with the data processing system 200 and accessed by the processing units 230 of one or more of the computers 210. The computing device stores 250 thereon or retains therein a program module 300 that includes computer program instructions which when executed by one or more of the processing units 230, performs a programmatically executable process for generating video clip imagery of the relative movement of one or more limbs about a joint sensed by sensors on the joint brace. Specifically, the program instructions during execution receive from over the data communications network 240 a set of sensor data 215 from a corresponding one of the remote clients 290 in connection with a particular time stamp and stores the sensor data 215 in fixed storage 215.

In response, the program instructions generate a visualization of the position of different limbs relative to a joint from positional information in the sensor data 215. The program instructions then submit a sequence of the visualizations for a corresponding one of the smart braces 270 to clip generator 225 and the program instructions receive in return, a video clip visualizing a movement of the limbs relative to the joint over a specified period of time defined by the time stamps of the visualizations. Subsequently, the program instructions query user table 235 to identify a network endpoint of a health care practitioner registered for the corresponding one of the smart braces 270. Finally, the program instructions transmit a message with the video clip over the data communications network 240 to a health care provider terminal 280 of the health care practitioner registered for the corresponding one of the smart braces 270.

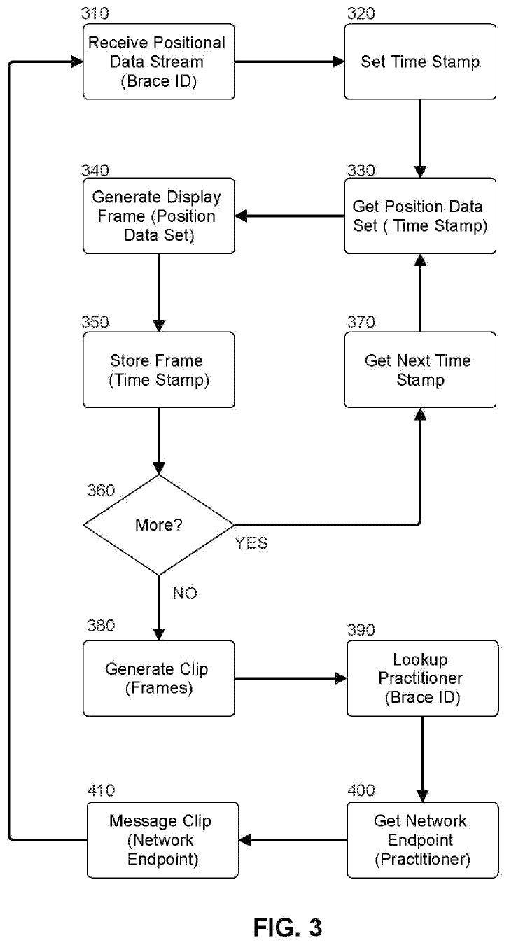

In further illustration of an exemplary operation of the module, FIG. 3 is a flow chart illustrating one of the aspects of the process of FIG. 1. Beginning in block 310, a positional data stream is received in memory in connection with an identifier for a specific brace. In block 320, a first time stamp in a chronological sequence of time stamps is established for the positional data stream, for instance the earliest time stamp present in connection with positional data in the stream. In block 330, a position data set is extracted from the stream for the time stamp and in block 340 a visualization is created visualizing a relative position of each of two limbs opposite a joint associated with the positional data stream. Thereafter, in block 350 the visualization is stored in connection with the time stamp.

In decision block 360, it is determined if more time stamps of the positional data stream have yet to be accounted for and if not, in block 370 a next time stamp in the chronological sequence of time stamps is determined. The process then repeats through block 330. In decision block 370, when no additional time stamps in the chronological sequence of time stamps have associated therewith unprocessed ones of the positional data stream, in block 380 a video clip is generated from the different visualizations. Then, in block 390 a health care practitioner is identified as having been registered in connection with an end user of the specified brace. Consequently, in block 300 a network endpoint such as an e-mail address is determined for the health care practitioner. Finally, the video clip is messaged to the network endpoint.

Of import, the foregoing flowchart and block diagram referred to herein illustrate the architecture, functionality, and operation of possible implementations of systems, methods, and computing devices according to various embodiments of the present invention. In this regard, each block in the flowchart or block diagrams may represent a module, segment, or portion of instructions, which includes one or more executable instructions for implementing the specified logical function or functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

More specifically, the present invention may be embodied as a programmatically executable process. As well, the present invention may be embodied within a computing device upon which programmatic instructions are stored and from which the programmatic instructions are enabled to be loaded into memory of a data processing system and executed therefrom in order to perform the foregoing programmatically executable process. Even further, the present invention may be embodied within a data processing system adapted to load the programmatic instructions from a computing device and to then execute the programmatic instructions in order to perform the foregoing programmatically executable process.

To that end, the computing device is a non-transitory computer readable storage medium or media retaining therein or storing thereon computer readable program instructions. These instructions, when executed from memory by one or more processing units of a data processing system, cause the processing units to perform different programmatic processes exemplary of different aspects of the programmatically executable process. In this regard, the processing units each include an instruction execution device such as a central processing unit or “CPU” of a computer. One or more computers may be included within the data processing system. Of note, while the CPU can be a single core CPU, it will be understood that multiple CPU cores can operate within the CPU and in either instance, the instructions are directly loaded from memory into one or more of the cores of one or more of the CPUs for execution.

Aside from the direct loading of the instructions from memory for execution by one or more cores of a CPU or multiple CPUs, the computer readable program instructions described herein alternatively can be retrieved from over a computer communications network into the memory of a computer of the data processing system for execution therein. As well, only a portion of the program instructions may be retrieved into the memory from over the computer communications network, while other portions may be loaded from persistent storage of the computer. Even further, only a portion of the program instructions may execute by one or more processing cores of one or more CPUs of one of the computers of the data processing system, while other portions may cooperatively execute within a different computer of the data processing system that is either co-located with the computer or positioned remotely from the computer over the computer communications network with results of the computing by both computers shared therebetween.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “include”, “includes”, and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

I claim:

1. A black box joint brace comprising:
a flexible sleeve defining a hollow channel with a circumference that is equal to or less than a circumference of a target limb encapsulating a mammalian joint;
a power source fixed to the sleeve;
a set of positional sensors disposed opposite to one another on the sleeve and sensing a spatial position of each of different limbs positioned oppositely across the mammalian joint; and,
memory powered by the power source and storing the spatial position sensed by the set of positional sensors.

2. The brace of claim 1, wherein the spatial position is a position of a lower one of the limbs relative to the mammalian joint.

3. The brace of claim 1, wherein the spatial position is a position of an upper one of the limbs relative to the mammalian joint.

4. The brace of claim 1, wherein the spatial position is a position of a lower one of the limbs relative to an upper one of the limbs.

5. The brace of claim 1, further comprising a rotary motion sensor powered by the power source and adapted to measure position, velocity and acceleration and to write the measured position, velocity and acceleration to the memory.

6. The brace of claim 1, further comprising a pulsometer fixed to an interior portion of the flexible sleeve and powered by the power source and adapted to measure a pulse through skin contact and to write the pulse to the memory.

7. The brace of claim 1, further comprising a temperature sensor fixed to an interior portion of the flexible sleeve and powered by the power source and adapted to measure of a temperature of the mammalian joint through skin contact and to write the temperature to memory.

8. The brace of claim 1, further comprising:
an embedded computing system powered by the power source; and,
firmware executing in the embedded computing system and including program instructions to read the sensed spatial position from the set of positional sensors and to write the sensed spatial position to the memory.

9. The brace of claim 1, wherein the mammalian joint is a knee joint.

10. The brace of claim 1, wherein the mammalian joint is an elbow joint.

11. The brace of claim 1, wherein the flexible sleeve is a neoprene sleeve.

12. The brace of claim 8, further comprising a shortwave radio frequency transmitter controlled by the embedded computing system and configured to transmit the spatial position sensed by the set of positional sensors to a coupled computing device.

13. The brace of claim 12, wherein the coupled computing device is a smart watch.

14. The brace of claim 12, wherein the coupled computing device is a mobile phone.
